(19) United States
(12) Patent Application Publication
(10) Pub. No.: US 2015/0245780 A1
(43) Pub. Date: Sep. 3, 2015

(54) METHOD AND APPARATUS FOR IDENTIFYING HYPERGLYCAEMIA

(71) Applicant: UNIVERSITY OF TECHNOLOGY, SYDNEY, Sydney, New South Wales (AU)

(72) Inventor: Hung Nguyen, Ultimo, NSW (AU)

(21) Appl. No.: 14/426,633

(22) PCT Filed: Aug. 29, 2013
(86) PCT No.: PCT/AU2013/000970

§ 371 Date: Mar. 6, 2015

(30) Foreign Application Priority Data
Aug. 29, 2012 (AU) 2012903747

(51) Int. Cl. A61B 5/04; A61B 5/0468; A61B 5/00; A61B 5/0245; A61B 5/024; A61B 5/145; A61B 5/0456

(52) U.S. Cl. CPC A61B 5/04012; A61B 5/14532; A61B 5/0468; A61B 5/0456; A61B 5/0245; A61B 5/02405; A61B 5/746; A61B 5/7405; A61B 5/742

(57) ABSTRACT

A method and apparatus for determining hyperglycaemia. The apparatus includes a sensor and a processor, to determine a QT interval of a patient and any rate of change of the QT interval (dQT/dt), and, output means, to provide an output signal indicative of hyperglycaemia using an algorithm based on the combination of the QT interval and the rate of change of the QT interval. The apparatus may further include further sensor means, to determine a TpTec parameter and the SDNN parameter of a patient, whereby the processor further determines any rate of change of the TpTec parameter (dTpTec/dt) and the SDNN parameter to provide an output signal based on any one or combination of the QT interval, the TpTec parameter, the SDNN parameter and the rate of change of these parameters.

### FIELD OF THE INVENTION

The present invention relates to a method and apparatus for identifying hyperglycaemia, and in particular to a method and apparatus to sense physiological responses of a patient from parameters of an ECG tracing of a patient for early detection of hyperglycaemic conditions.

### BACKGROUND OF THE INVENTION

The reference in this specification to any prior publication (or information derived from it), or to any matter which is known, is not, and should not be taken as, an acknowledgement or admission or any form of suggestion that prior publication (or information derived from it) or known matter forms part of the common general knowledge in the field of endeavour to which this specification relates.^{,}

Hyperglycaemia is a condition characterised by abnormally high blood glucose levels. It can lead to ketoacidocis which could be fatal.

On the other hand, hypoglycaemia is the most common complication experienced by patients with Type 1 diabetes. If not treated properly, severe hypoglycaemia may result in coma and irreversible brain damage.

For the purpose of detecting various classes of glycaemia, blood glucose levels (BGL) are used. Hypoglycaemia is often considered to be encountered when BGL<3.33 mmol/l, normoglycaemia is when 3.33 mmol/l<BGL<8.33 mmol/l, and, the hyperglycaemic state is present when BGL>8.33 mmol/l. Sometimes, hyperglycaemia is defined at different levels, for example BGL>11.1 mmol/l.

Conventionally, to determine hypoglycaemic or hyperglycaemic conditions, diabetic patients need to frequently monitor blood glucose level. One conventional technique, for example, requires that the patients draw blood, typically by pricking the finger. The drawn blood is then analysed by a portable device to determine blood glucose levels. The technique can be painful and therefore can significantly discourage the patient from periodically checking blood glucose levels. Obviously, non-invasive techniques would be very desirable.

Non-invasive methods proposed up to date include systems such as: infrared/near-infrared spectroscopy, iontophoresis, skin conductance, etc. However, none of these have proved sufficiently reliable or unobtrusive.

The inventor has, fairly recently, invented an effective and sensitive system to monitor hypoglycaemia non-invasively, using physiological parameters such as heart rate, skin impedance and electrocardiogram (ECG). This is disclosed in U.S. Pat. No. 7,450,986, the entire contents of which should be considered to be incorporated in this specification by their reference thereto. As described in U.S. Pat. No. 7,450,986, ECG offers a quicker, more ubiquitous, non-invasive clinical and research screen for the early detection of hypoglycaemia than other physiological signals.

In FIG. 1 is shown a typical ECG (Electrocardiograph) tracing of a cardiac cycle (heartbeat). The ECG tracing typically consists of a P wave, a QRS complex, and, a T wave.

The QT interval in particular reflects the duration of the intracellular action potential. It represents the time required for completion of both ventricular depolarisation and repolarisation. Recent studies indicate that insulin resistance affects the activation of the myocardium and can increase the QT interval. Because QT interval is influenced by chronotropic changes, Bazett defined the corrected QT interval (QTc), which is the measure generally used. QTc interval represents an index of myocardial refractoriness and electrical stability and it is associated with ventricular fibrillation and sudden cardiac death.

Other important ECG parameters include the interval from the peak of the T wave to its end (TpTe) and the associated corrected TpTe (TpTec), and, the standard deviation of the RR interval index (SDNN). The TpTe interval is suggested as a transmural dispersion index of repolarisation. A number of studies show that TpTe interval increases in patients with QT interval prolongation. In addition, the ratio of TpTe interval to QT interval is a potentially significant index for an arrhythmic event. These results imply that TpTe and QT intervals are important parameters contributing to ventricular repolarisation. On the other hand, SDNN is considered to reflect both sympathetic and parasympathetic influence on heart rate variability (HRV).

### SUMMARY OF THE INVENTION

The present invention seeks to provide a non-invasive method and apparatus of identifying hyperglycaemic conditions in patients.

The present invention also seeks to provide a method and apparatus for detecting hyperglycaemia which is relatively accurate, easy and inexpensive to use.

The present invention also seeks to provide a hyperglycaemia detection apparatus which may, in a preferred form, trigger an alarm signal within an acceptable time delay from when this condition presents itself, such that appropriate remedial action may be taken in a timely manner.

In one broad form, the present invention provides a method of determining hyperglycaemia, including the steps of:

sensing a QT interval of a patient;

determining any rate of change of said QT interval; and,

providing an output signal indicative of hyperglycaemia in the event that said rate of change of said QT interval reduces by a predetermined amount.

Preferably, the method further includes any one or combination of the steps of:

sensing a TpTec interval of a patient; and,

determining any rate of change of said TpTec interval;

whereby, said output signal is provided to indicate hyperglycaemia based on a correlation between said rate of change of each of said QT interval and said TpTec interval.

Also preferably, the method further includes the steps of:

sensing an RR interval of a patient;

determining the standard deviation of the RR interval (SDNN); and,

said output signal being provided to indicate hyperglycaemia in the event Of said standard deviation (SDNN) being above a predetermined value.

In a further broad form, the present invention provides a method of determining hyperglycaemia, including the steps of:

sensing at least one parameter of heart rate, QT interval, TpTec and SDNN_{;}

determining any rate of change of said parameter(s); and,

providing an output signal in the event of said rate of change of said parameter(s) being within a predetermined range.

In a further broad form, the present invention provides an apparatus for determining hyperglycaemia including:

a sensor;

and a processor, to determine a QT interval of a patient and the rate of change of said QT interval;

output means, to provide an output signal indicative of hyperglycaemia in the event that said rate of change of said QT interval reduces by a predetermined amount.

Preferably, the apparatus further determines the TpTec parameter of a patient;

wherein, said processor further determines any rate of change of said TpTec parameter, and

wherein, said output signal is provided based on a correlation between said rate of change of said QT interval and said TpTec parameter.

Preferably, the apparatus further determines the SDNN of the RR interval of a patient;

wherein, said processor further determines any rate of change of said SDNN parameter, and

wherein, said output signal is provided based on a correlation between said rate of change of any one or combination of said QT interval, said TpTec parameter, and/or said SDNN parameter.

In a further broad form, the present invention provides an apparatus for determining hyperglycaemia including:

a sensor, to sense at least one parameter, including heart rate, QT interval, TpTe, and/or SDNN;
a processor, to determine any rate of change of said parameter(s);
output means, to provide an output signal in the event of said rate of change of said parameter(s) being within a predetermined range.

Preferably, said processor includes a neural network to receive data obtained from said sensor(s), said neural network being programmed with an optimal learning algorithm.

Also preferably, said neural network is programmed with an optimal Bayesian network.

Also preferably, said output means includes an audio and/or visual alarm.

### BRIEF DESCRIPTION OF THE DRAWINGS

The present invention will become more fully understood from the following detailed description of preferred but non-limiting embodiments thereof, described in connection with the accompanying drawings, wherein:

FIG. 1(a) shows an ECG of a normal sinus rhythms, FIG. 1(b) shows an ECG of a patient with a high BGL, and FIG. 1(c) shows an ECG of a patient with a normal BGL;

FIG. 2 shows typical changes in ECG parameters under hyperglycaemic conditions;

FIG. 3 shows a flowchart describing the hyperglycaemia detection method/system of the present invention;

FIG. 4 shows the blood glucose profiles of five type 1 diabetes (T1D) patients;

FIG. 5 shows the evidence framework for Bayesian inference; and

FIG. 6 shows how the components may be typically attached to a patient.

### DETAILED DESCRIPTION OF THE INVENTION

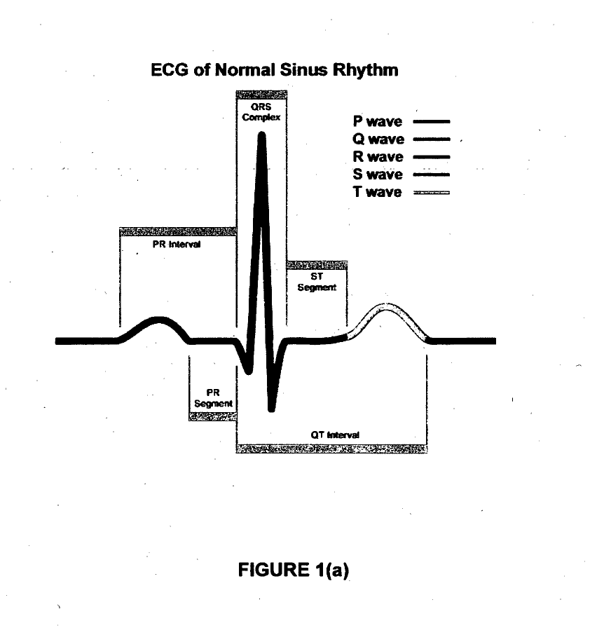

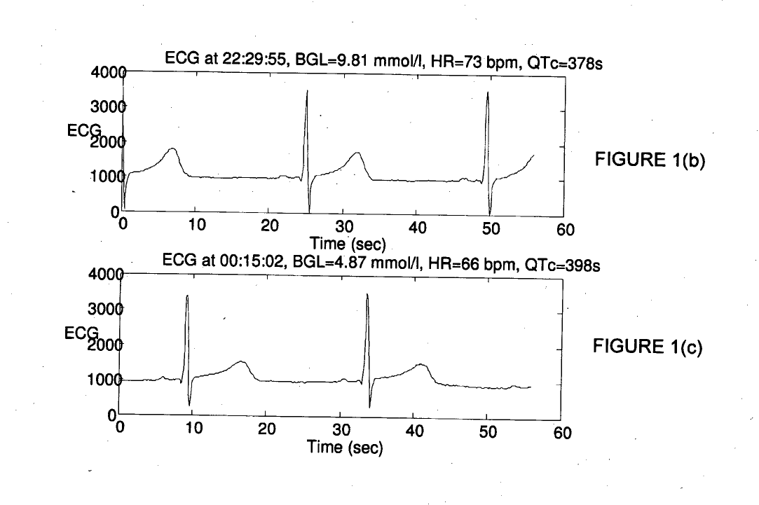

As previously described, in FIG. 1(a) is shown a typical ECG (Electrocardiograph) tracing of the cardiac cycle (heartbeat).

The inventor has identified that the onset of a hyperglycaemic condition results in changes to the ECG signal.

For comparison, FIG. 1(b) shows the ECG of a patient having a high blood glucose level (BGL) of 9.81 mmol/l, whilst FIG. 1(c) shows the ECG of the patient with a normal BGL of 4.87 mmol/l.

In particular, the inventor has identified that analysis of the effectiveness of ECG (in particular heart rate, QT interval, TpTe and SDNN) by means of an optimal neural network provides a novel basis for early detection of hyperglycaemia, as well as an indirect measurement of blood glucose levels. There are numerous factors which can affect the accuracy of the device such as environment conditions, stress, and the like. The device is capable of differentiating between effects caused by environmental conditions and those which initiate the presence of or onset of a particular medical condition.

The possibility of experimental hyperglycaemia has been shown to shorten QT intervals in both non-diabetic subjects and in those with Type 1 and Type 2 diabetes. It is envisaged that a suitable device may be used for the detection of conditions such as hyperglycaemia, or may be used to provide indirect measurement of blood glucose levels.

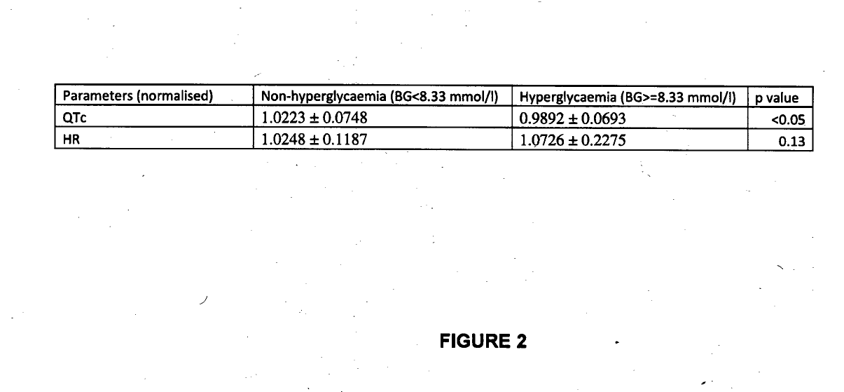

FIG. 2 shows typical changes in ECG parameters under hyperglycaemic conditions. In a hyperglycaemic state, an increase in PR is noted, a significant decrease in QTc, RTc, TpTec and SDNN are noted, but no significant changes in HR are noted. The present invention therefore provides a method and apparatus for effectively sensing these parameters, processing these sensed signals and providing an appropriate output, such that appropriate remedial action may be thereby taken.

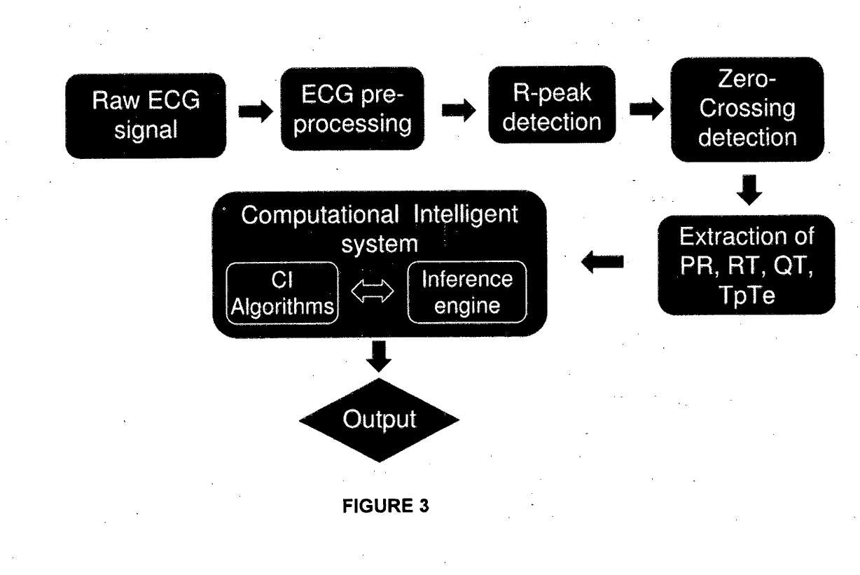

FIG. 3 shows a flowchart describing the hyperglycaemia detection method/process of the present invention, which provides an output signal based on a correlation of the ECG parameters and their rates of change.

There are many different ways to implement the signal sensing and signal conditioning for the device. One implementation strategy can be described as follows.

The ECG may be achieved by placing three Ag-AgCl electrodes in a LeadII configuration on the patient's chest. The signal obtained from the electrodes may then be amplified using an instrumentation amplifier with gain of 10 and CMRR>100 dB at 100 Hz. This feeds through a high-pass filter with cutoff frequency of 0.5 Hz. A second stage non-inverting amplifier may be added to provide a gain of 100. To obtain a reliable heart rate of the patient, a bandpass filter may be used to detect the QRS complex of the ECG signal. A threshold circuit together with a comparator may be used to reliably detect the R slope. The QT interval, on the other hand is a clinical parameter which can be derived from the ECG signal. Whilst it has been previously identified that during hypoglycaemia, the normalised QTc interval increases, the inventor has now also found that during hyperglycaemia, the normalised QTc interval decreases. QT measurement requires the identification of the start of QRS complex and the end of the T wave. The intersection of the isoelectric line and a tangent to the T wave can be used to measure the QT interval.

The monitoring for hyperglycaemia and blood glucose level non-invasively is difficult due to imperfections caused by possible conflicting or reinforcing responses from various ECG parameters. This conflicting information is preferably handled in the framework of an optimal Bayesian network in order to obtain accurate determinations from a complex uncertain non-linear physiological system.

For hyperglycaemia detection using a combination of one or more certain variables (heart rate, QT interval, TpTe, and standard deviation of the RR interval index (SDNN)), a computational intelligence method of analysis is suitable. A Bayesian network is suitable for controlling complex systems. This neuro-estimator may be embedded in an EEPROM of the system to monitor hyperglycaemia episodes in patients. This neural network has a multilayer feedforward neural network structure with one input layer, one hidden layer and one output layer. Essentially, this neural network is trained using a learning algorithm, in which synaptic strengths are systematically modified so that the response of the network will increasingly approximate the blood glucose status given by the available qualitative data.

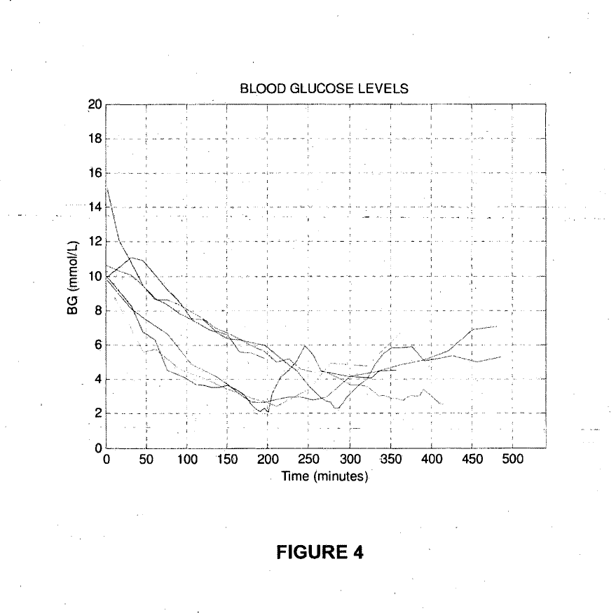

The inventor has tested responses from five T1D patients, and identified significant changes during the hyperglycaemia phase against the, non-hyperglycaemia phase. The actual blood glucose profiles are shown in FIG. 4.

This study shows that associated with hyperglycaemic episodes in 5 T1D patients, their normalised QTc interval reduced significantly (1.0223±0.0748 vs. 0.9892±0.0693, P<0.05). In addition, their TpTec interval and SDNN also reduced significantly (TpTec: 95.08±9.36 ms vs. 104.87±12.29 ms, P<0.0001, SDNN: 45.5±15.1 ms vs. 74.1±27.9 ms, P<0.0001). On the other hand, their heart rate HR increases, but not significantly (1.0248±0.1187 vs. 1.0726±0.2275, P=0.13).

Similar to the above ,solution, it is also possible to develop a Bayesian network for the classification of hyperglycaemia. In order to detect hyperglycaemic episodes reliably, it is not a simple matter of just using a combination of the above-mentioned parameters: heart rate, QT interval, TpTe and/or SDNN. The main difficulty is different patients have different base values of these parameters. In addition, these base values may vary from day to day.

False detection may arise from other conditions which could cause similar variations in QTc, TpTe, and/or SDNN. Avoidance of false detection is important if the system is to be relied on by T1D patients.

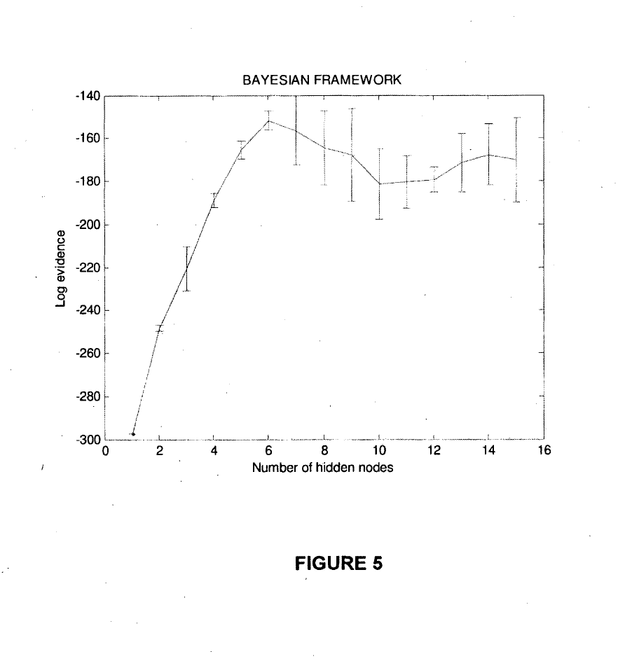

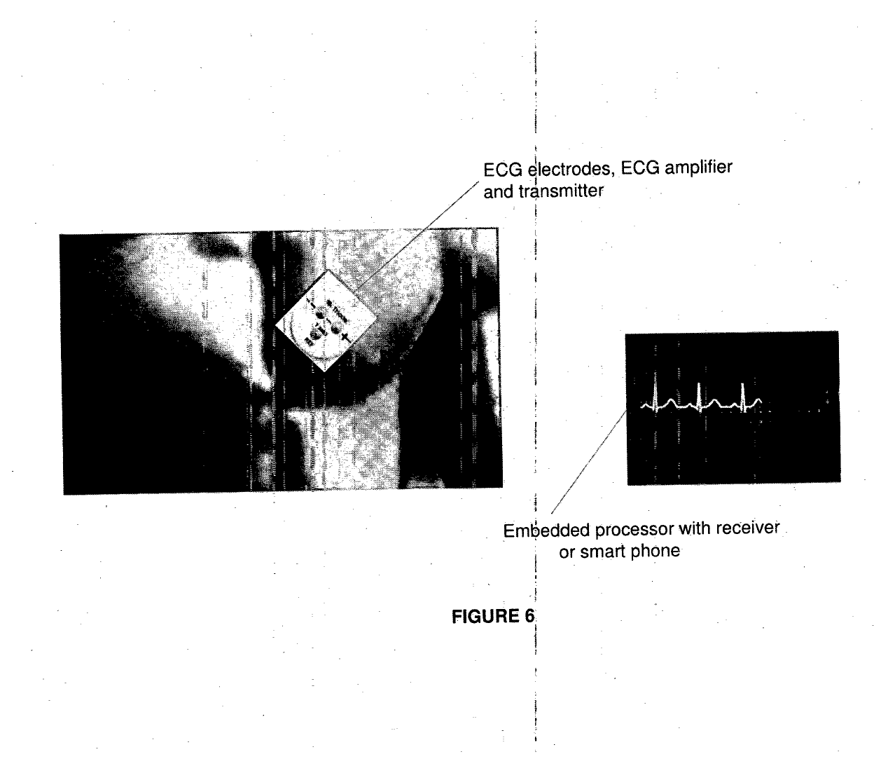

The overall data set consisted of a training set and a test set. For these, the whole data set which included both hyperglycaemia data part and non-hyperglycaemia data part were used. For optimal robustness of the evaluation, the framework for Bayesian inference was applied to the training set and it was found that the feedforward neural network architecture with 6 hidden nodes yielded the highest evidence, as shown in FIG. 5.

From the neural network which was derived from the training set with the highest log evidence, estimated blood glucose profiles were found to be correlated significantly to the actual blood glucose values obtained for the test set (r=0.408, P<0.0002). In addition, the predicted hyperglycaemia classifications in the test set were found to be correlated to the, actual hyperglycaemic episodes (r=0.561, P<0.0001). From the optimal neural network which was derived from the training set, the sensitivity (true positive) value and the specificity (true negative) for the detection of hyperglycaemia (BG>8.33 mmol/l) in the test set are 80% and 56% respectively. This has been achieved across various sleep stages.

Communication between the sensors and the processor may be via a telemetric system, with radio frequency transmitter and receivers at typically 2.4 GHz). Other appropriate communication systems will be apparent to persons skilled in the art.

The output may be provided in any appropriate format, such as an alarm or other visual or audible output. The alarm may be of any convenient type, and may include a simple radio alarm, a signal transmitted to a monitoring station, or the like.

The data transmitted from the sensors may either be continuously logged, or monitored at appropriate discrete (short) intervals.

The system may be typically interfaced with a PC which will continuously log the, relevant data using a data management system such as Labview.

Clearly the invention can vary from that described herein without departing from the scope of the invention. In particular the neural network algorithm needs not be of the type described herein, but any optimal neural network algorithm that is able to provide substantially real time analysis of multiple data streams in the manner described herein could be used.

It will therefore be appreciated that the present invention provides a non-invasive method of determining the presence or onset of the hyperglycaemic condition in a person. This method includes, continuously monitoring at least one or more ECG trace parameters of the patient; including, but not limited to heart rate, QTc interval, TpTec interval, and, standard deviation of the RR interval index (SDNN). It then establishes whether one or more of those monitored parameters changes, and if so, the rate of change of that parameter or parameters.

Data obtained in the first two steps is preferably fed into a neural network programmed with an optimal algorithm. An output signal, such as an alarm signal may be triggered when said neural network establishes conditions which suggest the presence or imminent onset of said hyperglycaemic condition.

The monitoring of the heart rate, QT interval, TpTe and/or SDNN is preferably done with an ECG. The optimal learning algorithm may be based on a Bayesian neural network.

The invention extends to apparatus for generating an alarm or other output when a hyperglycaemic condition is present or imminent in a person. The apparatus includes sensors for sensing at one or more of the heart rate, QT interval, TpTe, and SDNN. One or more of the parameters is monitored for change, and, the rate of its change.

A neural network linked to said sensors may, for example, receive a substantially continuous data stream from said sensors. The neural network is programmed with an optimal learning algorithm and adapted to establish when the sensed parameters, and any change to those parameters, for a particular person are such as to indicate the presence or imminent onset of the physiological condition.

An alarm or other output linked to said neural network is adapted to be triggered when the presence or imminent onset of said hyperglycaemic condition is established.

The apparatus may include an optimal Bayesian network.

The present invention therefore provides a method and apparatus for detecting a reduction in QT interval, monitoring its rate of change (dQT/dt) in a patient, and, if the rate of change reduces by a predetermined amount, provides an output signal which indicates a hyperglycaemic condition in the patient.

The present invention also monitors for change in TpTe parameters, monitors any rate of change (dTpTe/dt) and, likewise, is processed to provide a corresponding output signal.

The present invention also monitors for change in the standard deviation of the RR interval (SDNN), and, likewise is also processed to provide a corresponding output signal.

All such variations and modifications which become apparent to persons skilled in the art should be considered to fall within the scope of the present invention as broadly hereinbefore described.

1. A method of determining hyperglycaemia, including the steps of:
sensing and determining a QTc interval of a patient;
determining any rate of change of said QTc interval (dQTc/dt); and,
providing an output signal indicative of hyperglycaemia based on said QTc interval and said rate of change of said QTc interval.

2. A method of determining hyperglycaemia as claimed in claim 1, further including any one or combination of the steps of:
determining a TpTe_{c} interval of a patient; and
determining any rate of change of said TpTec interval (dTpTec/dt);
determining the standard deviation of the RR interval (SDNN); and
determining any rate of change of said SDNN interval (dSDNN/dt);
said output signal being provided to indicate hyperglycaemia based on any one or combination of said QTc interval, TpTec interval, and SDNN and/or said rate of change of these said parameters.

3. A method of determining hyperglycaemia, including the steps of:
sensing at least one parameter of heart rate, QTc interval, TpTec and/or SDNN;
determining any rate of change of said parameter(s); and,
providing an output signal based on any one or combination of these said parameters and their rates of change.

4. An apparatus for determining hyperglycaemia including:
a sensor, to sense a QTc interval of a patient;
a processor, to determine any rate of change of said QTc interval (dQTc/dt); and,
output means, to provide an output signal indicative of hyperglycaemia based on the combination of said QTc interval and said rate of change of said QTc interval.

5. An apparatus for determining hyperglycaemia as claimed in claim 4, further including:
a sensor to sense a TpTec parameter of a patient;
wherein, said processor further determines any rate of change of said TpTec parameter (dTpTec/dt), and
wherein, said output signal is provided based on any one or combination of said QTc interval, TpTec interval and said rate of change of said QT interval and said TpTec parameter.

6. An apparatus for determining hyperglycaemia as claimed in claim 6, further including:
a sensor to sense an RR interval of a patient;
wherein said processor determines a standard deviation (SDNN) of the RR interval; and
wherein, said output signal is provided based on any one or combination of said QTc interval, TpTec interval, SDNN and said rate of change of these said parameters.

7. An apparatus for determining hyperglycaemia including:
a sensor, to sense at least one parameter of heart rate, QT interval, TpTe and/or SDNN;
a processor, to determine any rate of change of said parameter(s);
output means, to provide an output signal using any one or combination of said parameters and said rate of change of said parameter(s).

8. An apparatus for determining hyperglycaemia as claimed in claim 5, wherein
said processor includes a neural network to receive data obtained from said sensor(s), said neural network being programmed with an optimal learning algorithm.

9. An apparatus as claimed in claim 8, wherein said neural network is programmed with an optimal Bayesian network.

10. An apparatus as claimed in claim 4, wherein said output means includes an audio and/or visual alarm.
